(19) United States
(12) Patent Application Publication
(10) Pub. No.: US 2026/0183443 A1
(43) Pub. Date: Jul. 2, 2026

(54) LIQUID STORAGE BOTTLE AND FRAGRANCE DIFFUSER

(71) Applicant: Guangdong Chiyang Scent Technology Co ., LTD., Foshan (CN)

(72) Inventors: Xiaoyang Gao, Foshan (CN); Qingteng Huang, Foshan (CN); Qunqing Zhu, Foshan (CN)

(21) Appl. No.: 19/208,620

(22) Filed: May 15, 2025

(30) Foreign Application Priority Data
Dec. 31, 2024 (CN) 202423322255.8

(51) Int. Cl. A61L 9/14; B65D 1/02

(52) U.S. Cl. CPC A61L 9/14; B65D 1/0246; A61L 2209/133; A61L 2209/134; A61L 2209/14

(57) ABSTRACT

A liquid storage bottle includes: a bottle body, defining a bottle mouth and a liquid storage chamber; and a bottle plug, sealing the bottle mouth and integrating an atomizing core, a liquid sucking tube and a connecting component. The atomizing core defines a liquid conveying channel and an air conveying channel respectively connected to the liquid sucking tube and the connecting component. The connecting component is connected to an external air supply device. Liquid fragrance in the liquid storage chamber is sucked and conveyed to the atomizing core by the liquid sucking tube, mixed with the air flow from the connecting component and atomized, and then output through a spray outlet channel of the bottle plug. This design is compact and user-friendly. Due to low cost and relatively short lifespan, the atomizing core is inside the bottle plug, making it convenient for disposal and replacement while ensuring the atomization effect.

### TECHNICAL FIELD

The disclosure relates to the field of fragrance technologies, and more particularly to a liquid storage bottle and a fragrance diffuser.

### BACKGROUND

A Chinese patent literature with publication No. CN217793904U discloses an aroma diffuser with a structure including a body, an atomizing head, and an air pump as key components. The body is provided with a spray nozzle inside, surrounded by multiple air nozzles connected to internal air paths. The atomizing head integrates an atomizing seat, an atomizing core, and an atomizing cover. The atomizing core is connected to air and liquid paths, which are connected to the air pump and a liquid storage bottle, respectively. The spray nozzle is located on the atomizing cover, and the atomizing seat is covered by a cover body, defining multiple independent chambers: an atomizing chamber, a first chamber, and a second chamber. A fan is connected to the first and second chambers to promote air circulation. A side wall of the atomizing seat defines a ventilation opening, while the cover body defines a through-hole that aligns with the spray nozzle. The air nozzles are connected to the second chamber, enabling a long-range spray effect.

At present, the design of the atomizing head in the diffuser has significant user operation risks and performance issues. The atomizing head is directly fitted onto a bottle mouth of the essential oil bottle, which is convenient for installation and removal and for users to replace essential oil, while resulting in potential hazards. Since the users often struggle to assess the quality of the essential oil they purchase, the essential oil may contain impurities or have inconsistent viscosities. Long-term use of such essential oil can not only affect the atomizing performance of the atomizing head but also lead to clogging of the atomizing core, impacting the overall performance and lifespan of the diffuser.

Moreover, the direct connection between the atomizing head and the essential oil bottle makes the bottle mouth fully exposed when the atomizing head is removed, which increases the risk of essential oil contamination, especially in dusty or humid environments. Once contaminated, the essential oil can not only affect the diffusion effect but also lead to potential health risks to the users.

### SUMMARY

In response to the related art, the disclosure provides a liquid storage bottle, the liquid storage bottle includes a bottle body and a bottle plug. The bottle body defines a bottle mouth and a liquid storage chamber, the bottle mouth is connected to the liquid storage chamber, and the liquid storage chamber is configured to store liquid fragrance. The bottle plug is inserted into the bottle mouth to form sealed fit therewith. The bottle plug is provided with an atomizing core, a liquid sucking tube and a connecting component. The atomizing core defines a liquid conveying channel and an air conveying channel. A liquid outlet hole of the liquid conveying channel and an air outlet hole of the air conveying channel are defined above a liquid surface. An input end of the connecting component is configured to be connected to an external air supply device, an output end of the connecting component is connected to the air conveying channel, and the connecting component is configured to provide an air flow to the atomizing core. An end of the liquid sucking tube is inserted into the liquid storage chamber, another end of the liquid sucking tube is connected to the liquid conveying channel, and the liquid sucking tube is configured to convey the liquid fragrance in the liquid storage chamber to the atomizing core. The atomizing core is configured to mix and atomize the air flow provided by the connecting component and the liquid fragrance provided by the liquid sucking tube. The bottle plug defines a spray outlet channel.

In an embodiment, the bottle plug is detachably inserted into the bottle mouth to form sealed fit therewith; or the bottle plug is provided with anti-dismantle components, the bottle body is provided with anti-dismantle parts around the bottle mouth, and when the bottle plug is inserted into the bottle mouth, the anti-dismantle components are engaged with the anti-dismantle parts.

In an embodiment, the bottle plug includes an upper plug body and a lower plug body, the lower plug body is inserted into the bottle mouth and in a sealing connection with the bottle mouth through a sealing ring, an outer wall of the bottle body around the bottle mouth is provided with outer threads, and the bottle body is fixed with an atomizing head at the bottle mouth through the outer threads; or
the bottle plug includes an inner plug body and an outer thread sleeve, the outer thread sleeve is sleeved on the outer wall of the bottle body around the bottle mouth, an outside of the outer thread sleeve is provided with outer threads, the outer thread sleeve is fixed with the atomizing head through the outer threads, the inner plug body is inserted into the bottle mouth, and upper ends of the inner plug body and the outer thread sleeve are connected to each other and are in a sealing connection with a top of the bottle body around the bottle mouth through the sealing ring.

In an embodiment, the bottle plug includes an inner plug body and an outer thread sleeve, the outer thread sleeve is sleeved on the outer wall of the bottle body around the bottle mouth, an inner wall of the outer thread sleeve is provided with anti-dismantle components, the outer wall of the bottle body around the bottle mouth is provided with anti-dismantle parts, the anti-dismantle components are disposed corresponding to the anti-dismantle parts, and the outer thread sleeve is fixed on the bottle body around the bottle mouth through an interference fit between the anti-dismantle components and the anti-dismantle parts.

In an embodiment, the inner wall of the outer thread sleeve is provided with limit components, the outer wall of the bottle body around the bottle mouth defines limit grooves, the limit components and the limit grooves extend along an axial direction of the bottle mouth, when the bottle plug is inserted into the bottle mouth, the limit components are inserted into the limit grooves respectively along the axial direction of the bottle mouth, and the limit components and the limit grooves are configured to limit rotating of the bottle plug relative to the bottle mouth.

In an embodiment, the anti-dismantle components and the anti-dismantle parts are protrusions disposed annularly, the anti-dismantle components extend inward along a radial direction of the bottle mouth, the anti-dismantle parts extend outward along the radial direction of the bottle mouth, a total length of one of the anti-dismantle components and a corresponding anti-dismantle part along the radial direction of the bottle mouth is greater than a distance between the inner wall of the outer thread sleeve and the outer wall of the bottle body around the bottle mouth, when the bottle plug is inserted into the bottle mouth, the anti-dismantle components are disposed under the anti-dismantle parts, the limit grooves penetrate through the anti-dismantle parts along the axial direction of the bottle mouth, and a width of each limit component is adapted to a width of a corresponding limit groove.

In an embodiment, the bottle plug includes a plug body which is tube-shaped, the plug body is in sealed fit with the inner wall of the bottle body around the bottle mouth, the connecting component is a connecting tube inserted into and connected to the plug body, the connecting component defines an installation through hole, the input end of the connecting component is configured to connect to the external air supply device in a plug-in manner, an air input end of the atomizing core is installed on an output end of the installation through hole in a plug-in manner, and the spray outlet channel is defined between an inner wall of the plug body and an outer wall of the connecting component.

In an embodiment, the connecting component is connected to the plug body through multiple connecting ribs, and the liquid outlet hole of the liquid conveying channel and the air outlet hole of the air conveying channel are disposed under the multiple connecting ribs. The spray outlet channel passes through the multiple connecting ribs, coaxially surrounds the connecting component, and connects an inside of the bottle body to an outside of the bottle body. The air outlet hole and the liquid outlet hole are spaced apart by a preset distance, axes of the air outlet hole and the liquid outlet hole intersect, and the air outlet hole is configured to blow the air flow from top to bottom to an outer end surface of the liquid outlet hole.

In an embodiment, the atomizing core is provided with a tube inserting sleeve, the another end of the liquid sucking tube is inserted into the tube inserting sleeve and connected to the liquid conveying channel, and the end of the liquid sucking tube is connected to a fragrance filter in a plug-in manner.

In another aspect, the disclosure provides a fragrance diffuser (also referred to as aroma diffuser). The fragrance diffuser includes an atomizing head and the liquid storage bottle described above. The atomizing head is provided with an air output end, the air output end is detachably connected to the connecting component, the atomizing head is provided with a connecting head, the connecting head is configured to connect to the external air supply device, and the connecting head is connected to the air conveying channel through the air output end and the connecting component.

In an embodiment, the atomizing head includes a shell, the shell defines a spray outlet chamber and a spray outlet nozzle; the spray outlet nozzle is connected to the spray outlet chamber; an inside wall of the shell is provided with connecting threads, and an outer wall of the bottle body around the bottle mouth is provided with outer threads or an outer wall of the bottle plug is provided with outer threads to match with the connecting threads; the connecting component is an installation through hole defined on the bottle plug, the installation through hole is coaxial with the outer threads, the air output end is tube-shaped and coaxial with the connecting threads; when the connecting threads match with the outer threads, an end of the air output end is inserted into the installation through hole; the input end of the connecting component is configured to connect to the external air supply device in a plug-in manner; and an air input end of the atomizing core is tube-shaped and installed on an output end of the installation through hole in a plug-in manner.

In an embodiment, the liquid outlet hole of the liquid conveying channel and the air outlet hole of the air conveying channel are disposed in an inside of the bottle body, and the spray outlet channel is connected to the inside of the bottle body and the spray outlet chamber individually.

In an embodiment, the connecting head is elastic, an outer wall of the connecting head protrudes outward relative to the shell, the fragrance diffuser further includes an air pump, the connecting head abuts against an output end of the air pump, the air pump is configured to provide the air flow to the atomizing core through the connecting head, the air conveying channel penetrates through the air output end, an air inlet hole of the air conveying channel penetrates through the shell, and the connecting head is detachably installed on the air inlet hole in a plug-in manner to form sealed fit.

In an embodiment, the atomizing head further includes an atomizing filter, the atomizing filter is installed inside the spray outlet chamber, an input end of the atomizing filter is connected to the spray outlet chamber, a top of the shell is threadedly connected to a cover, the cover is configured to seal an opening of the shell, and the spray outlet nozzle is defined on the cover and connected to an output end of the atomizing filter.

In an embodiment, a top of the bottle plug is in a sealing connection with the shell through a sealing pad.

### BRIEF DESCRIPTION OF DRAWINGS

The above and other purposes, features, and advantages of the disclosure will become clearer through more specific explanations of illustrative embodiments shown in the accompanying drawings. Same reference signs in all the accompanying drawings indicate same parts, and the accompanying drawings are not intentionally scaled proportionally according to actual dimensions. The focus is on illustrating the main idea of the disclosure.

FIG. 1 illustrates a schematic structural diagram of a fragrance diffuser according to an embodiment of the disclosure.

FIG. 2 illustrates a schematic structural diagram of the fragrance diffuser illustrated in FIG. 1 from another perspective.

FIG. 3 illustrates a sectional view taken along a line A-A illustrated in FIG. 2.

FIG. 4 illustrates a partial structural diagram of the fragrance diffuser illustrated in FIG. 3.

FIG. 5 illustrates an exploded view of the fragrance diffuser illustrated in FIG. 1.

FIG. 6 illustrates a schematic diagram of an atomizing assembly according to the embodiment of the disclosure.

FIG. 7 illustrates a schematic diagram of a liquid storage bottle according to another embodiment of the disclosure.

FIG. 8 illustrates a sectional view taken along a line B-B illustrated in FIG. 7.

FIG. 9 illustrates an exploded view of a bottle body and a bottle plug illustrated in FIG. 7.

FIG. 10 illustrates a schematic diagram of the fragrance diffuser according to the embodiment of the disclosure.

Description of reference signs: 100: bottle body; 101: bottle mouth; 102: liquid storage chamber; 104: atomizing core; 105: liquid outlet hole; 106: air outlet hole; 107: liquid conveying channel; 108: air conveying channel; 109: bottle plug; 1011: outer thread; 1081: air inlet hole; 11: plug body; 110: upper plug body; 111: lower plug body; 112: spray outlet channel; 113: connecting component; 113a: connecting rib; 1131: installation through hole; 114: sealing ring; 115: tube inserting sleeve; 116: liquid sucking tube; 117: fragrance filter; 119: air input end; 200: atomizing head; 201: shell; 202: spray outlet nozzle; 203: spray outlet chamber; 204: connecting thread; 205: atomizing filter; 206: connecting head; 207: cover; 208: sealing pad; 209: air output end; 2011: opening; a01: anti-dismantle part; b01: limit groove; a09: anti-dismantle component; b09: outer thread sleeve; b091: outer thread; c09: limit component; d09: inner plug body; 300: air pump.

### DETAILED DESCRIPTION OF EMBODIMENTS

In order to facilitate the understanding of the disclosure, a more comprehensive description is provided below with reference to the relevant drawings.

It should be noted that when a component is considered to be “connected” to another component, the component can be directly connected to the another component and integrated with it, or there may be an intermediate component present at the same time. The terms “installation”, “one end”, “another end”, and similar expressions used herein are for illustrative purposes only.

Unless otherwise defined, all technical and scientific terms used herein have same meanings as those commonly understood by those skilled in the art. The terms used in this specification are only for the purpose of describing specific embodiments and are not intended to limit the disclosure. The term “and/or” used herein includes any and all combinations of one or more related listed items.

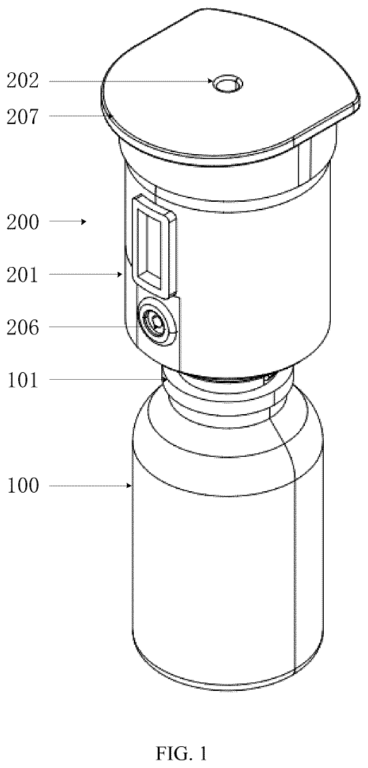

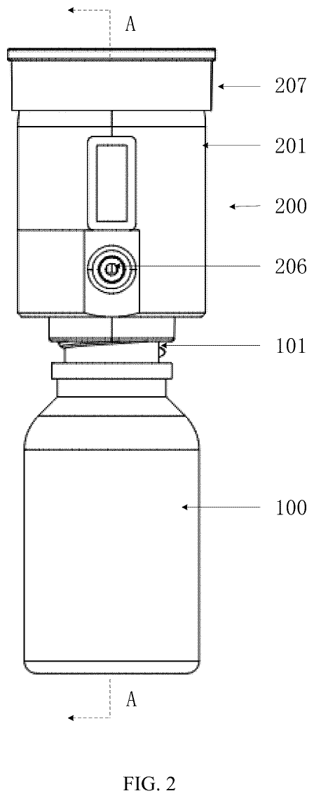

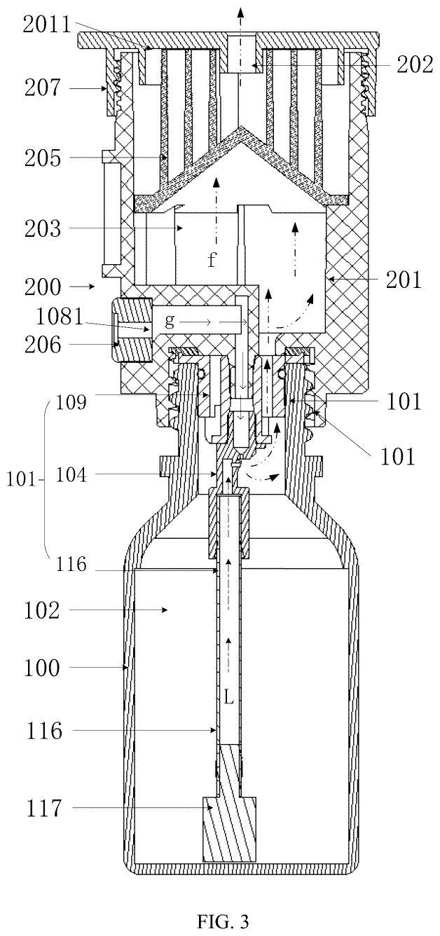

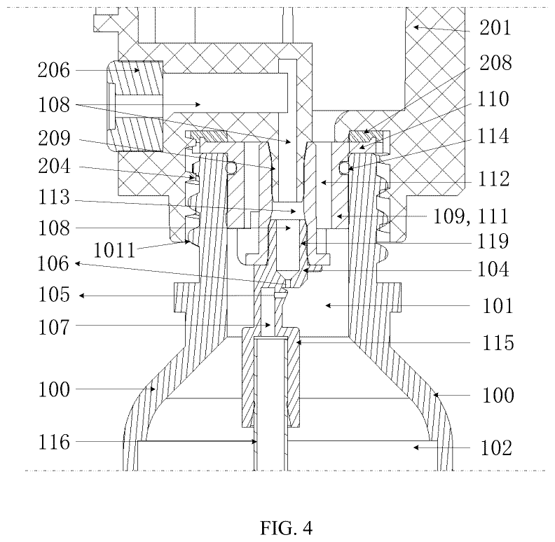

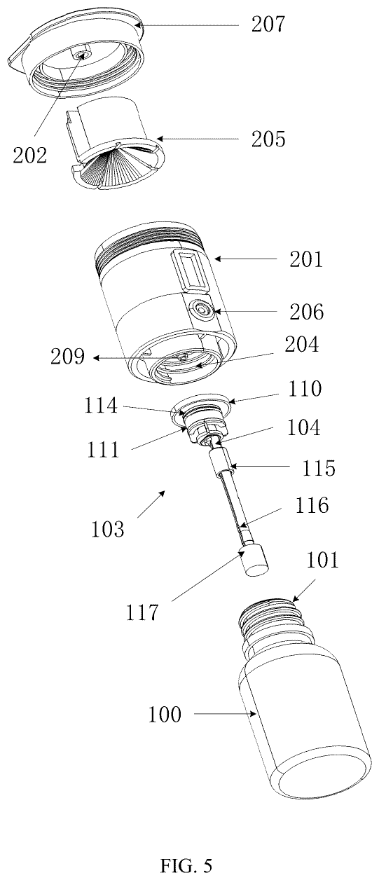

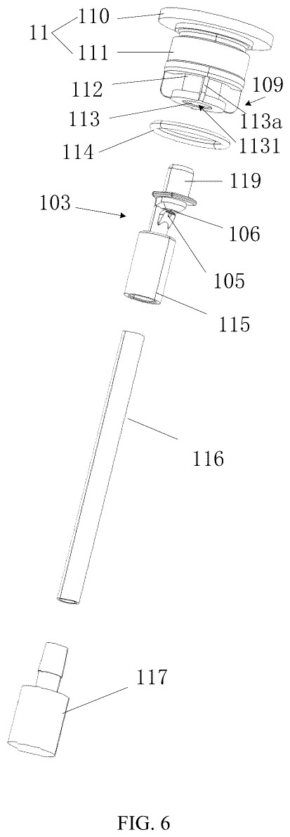

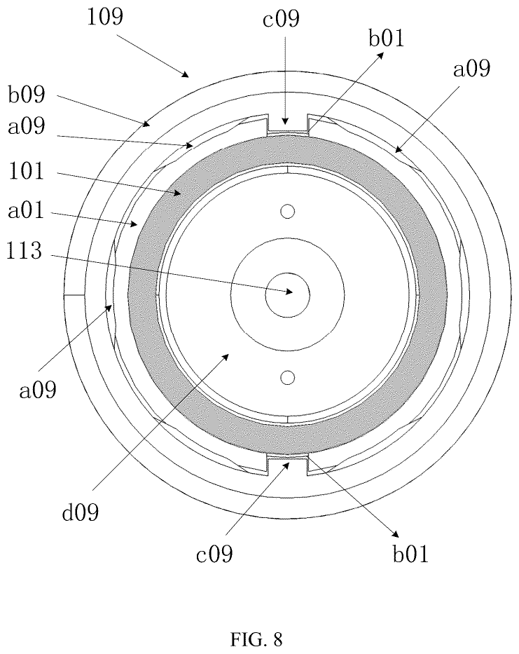

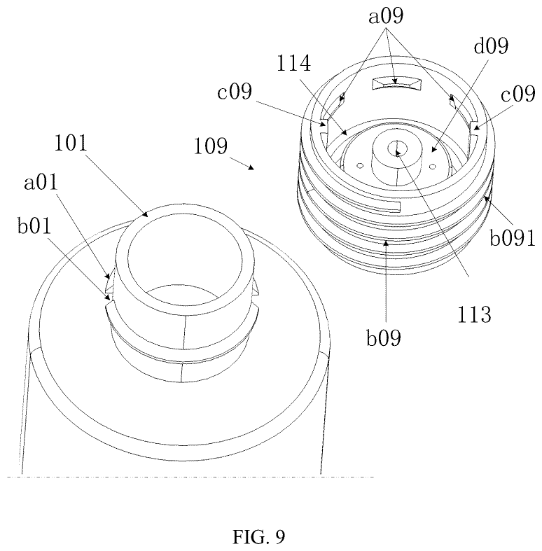

Referring to FIG. 1 to FIG. 9, in response to the related art, the disclosure provides a liquid storage bottle. The liquid storage bottle includes a bottle body 100 and a bottle plug 109. The bottle body 100 defines a bottle mouth 101 and a liquid storage chamber 102. The bottle mouth 101 is connected to the liquid storage chamber 102, and the liquid storage chamber 102 is configured to store liquid fragrance. The bottle plug 109 is inserted into the bottle mouth 101 to form sealed fit therewith. The bottle plug 109 is provided with an atomizing core 104, a liquid sucking tube 116 and a connecting component 113. The atomizing core 104 defines a liquid conveying channel 107 and an air conveying channel 108, and a liquid outlet hole 105 of the liquid conveying channel 107 and an air outlet hole 106 of the air conveying channel 108 are defined above a liquid surface. An input end of the connecting component 113 is configured to be connected to an external air supply device, an output end of the connecting component 113 is connected to the air conveying channel 108, and the connecting component 113 is configured to provide an air flow to the atomizing core 104. An end of the liquid sucking tube 116 is inserted into the liquid storage chamber 102, another end of the liquid sucking tube 116 is connected to the liquid conveying channel 107, and the liquid sucking tube 116 is configured to convey the liquid fragrance in the liquid storage chamber 102 to the atomizing core 104. The atomizing core 104 is configured to mix and atomize the air flow provided by the connecting component 113 and the liquid fragrance provided by the liquid sucking tube 116. The bottle plug 109 defines a spray outlet channel 112.

The atomizing core 104 defines the liquid conveying channel 107 and the air conveying channel 108, and the liquid outlet hole 105 of the liquid conveying channel 107 and the air outlet hole 106 of the air conveying channel 108 are defined above a liquid surface. The connecting component 113 is configured to be connected to the external air supply device and provide the air flow to the atomizing core 104. The liquid sucking tube 116 is configured to convey the liquid fragrance in the liquid storage chamber 102 to the atomizing core 104 to mix the liquid fragrance with the air flow followed by atomizing to obtain atomized fragrance. The atomized fragrance is output through the spray outlet channel 112. By setting the atomizing core 104, the liquid sucking tube 116 and the connecting component 113, the liquid fragrance is atomized and then output through the spray outlet channel 112. The liquid storage bottle is compact and easy to use. Due to the low cost and relatively short lifespan of the atomizing core 104, the atomizing core 104 is disposed in the bottle plug 109 and can be discarded along with the bottle after use. When replacing, a new atomizing core 104, a new bottle plug 109 and a new bottle body 100 can be used, which simplifies a replacement process and ensures optimal atomization performance.

Referring to FIG. 1 to FIG. 9, in a specific embodiment, the bottle plug 109 is detachably inserted into the bottle mouth 101 to form sealed fit therewith. Or, the bottle plug 109 is provided with anti-dismantle components a09, the bottle body 100 is provided with anti-dismantle parts a01 around the bottle mouth 101, and when the bottle plug 109 is inserted into the bottle mouth 101, the anti-dismantle components a09 are engaged with the anti-dismantle parts a01. The disclosure provides two installation method of the bottle plug 109 including a detachable plug-in type and an anti-dismantle type, which enhances the flexibility and security of the liquid storage bottle. The detachable plug-in type: the bottle plug 109 is fit with the bottle mouth 101 in a plug-in manner for easy replacement and maintenance. The anti-dismantle type: the bottle plug 109 is provided with the anti-dismantle components a09, the bottle body 100 is provided with the anti-dismantle parts a01 around the bottle mouth 101, the anti-dismantle components a09 are engaged with the anti-dismantle parts a01 to prevent the bottle plug 109 from being removed. If an attempt is made to forcefully remove the bottle plug 109, it will damage the seal between the bottle plug 109 and the bottle mouth 101, rendering the liquid storage bottle unusable.

Referring to FIG. 1 to FIG. 9, in a specific embodiment, the bottle plug 109 includes an upper plug body 110 and a lower plug body 111, the lower plug body 111 is inserted into the bottle mouth 101 and in a sealing connection with the bottle mouth 101 through a sealing ring 114, an outer wall of the bottle body 100 around the bottle mouth 101 is provided with outer threads 1011, and the bottle body 100 is fixed with an atomizing head 200 at the bottle mouth 101 through the outer threads 1011. Or the bottle plug 109 includes an inner plug body d09 and an outer thread sleeve b09, the outer thread sleeve b09 is sleeved on the outer wall of the bottle body 100 around the bottle mouth 101, an outside of the outer thread sleeve b09 is provided with outer threads b091, the outer thread sleeve b09 is fixed with the atomizing head 200 through the outer threads b091, the inner plug body d09 is inserted into the bottle mouth 101, and upper ends of the inner plug body d09 and the outer thread sleeve b09 are connected to each other and are in a sealing connection with a top of the bottle body 100 around the bottle mouth 101 through the sealing ring 114. The disclosure provides two sealing connection schemes between the bottle plug 109 and the bottle mouth 101, enhancing the seal and stability of the liquid storage bottle. In a first scheme, the lower plug body 111 is in a sealing connection with the bottle mouth 101 through the sealing ring 114, and the bottle body 100 is fixed with the atomizing head 200 at the bottle mouth 101 through the outer threads 1011. In a second scheme, the inner plug body d09 and the outer thread sleeve b09 are combined, the inner plug body d09 is inserted into the bottle mouth 101, the outer thread sleeve b09 is fixed with the atomizing head 200 through the outer threads b091 and in a sealing connection with the top of the bottle body 100 around the bottle mouth 101 through the sealing ring 114. When replacing fragrance, compared to the first scheme, the second scheme is more likely to encourage users to adopt a new set of matching components, including the liquid fragrance, the atomizing core 104, the bottle plug 109, and the bottle body 100.

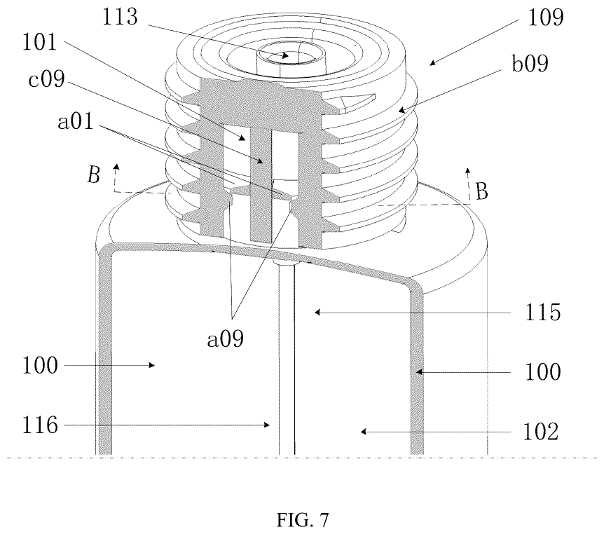

Referring to FIG. 7 to FIG. 9, in a specific embodiment, the bottle plug 109 includes the inner plug body d09 and the outer thread sleeve b09, the outer thread sleeve b09 is sleeved on an outer wall of the bottle body 100 around the bottle mouth 101, an inner wall of the outer thread sleeve b09 is provided with the anti-dismantle components a09, the outer wall of the bottle body 100 around the bottle mouth 101 is provided with the anti-dismantle parts a01, the anti-dismantle components a09 are disposed corresponding to the anti-dismantle parts a01, and the outer thread sleeve b09 is fixed on the bottle body 100 around the bottle mouth 101 through an interference fit between the anti-dismantle components a09 and the anti-dismantle parts a01. By the interference fit between the anti-dismantle components a09 and the anti-dismantle parts a01, the stability and robustness of the connection between the bottle plug 109 and the bottle mouth 101 are further enhanced. Furthermore, the anti-dismantle components a09 have a certain degree of elasticity to better achieve the interference fit.

Referring to FIG. 7 to FIG. 9, in a specific embodiment, the inner wall of the outer thread sleeve b09 is provided with limit components c09, the outer wall of the bottle body 100 around the bottle mouth 101 defines limit grooves b01, the limit components c09 and the limit grooves b01 extend along an axial direction of the bottle mouth 101, when the bottle plug 109 is inserted into the bottle mouth 101, the limit components c09 are inserted into the limit grooves b01 respectively along the axial direction of the bottle mouth 101, and the limit components c09 and the limit grooves b01 are configured to limit rotating of the bottle plug 109 relative to the bottle mouth 101. The limit components c09 and the limit grooves b01 extend along an axial direction of the bottle mouth 101, when the bottle plug 109 is inserted into the bottle mouth 101, the limit components c09 are inserted into the limit grooves b01, thereby achieving limit function. This arrangement restricts the rotation of the bottle plug 109 relative to the bottle mouth 101. When the atomizing head 200 is connected to the outer thread sleeve b09 through the outer threads b091, the outer thread sleeve b09 will not rotate with the atomizing head 200, improving the efficiency and accuracy of assembly

Referring to FIG. 7 to FIG. 9, in a specific embodiment, the anti-dismantle components a09 and the anti-dismantle parts a01 are protrusions disposed annularly, the anti-dismantle components a09 extend inward along a radial direction of the bottle mouth 101, the anti-dismantle parts a01 extend outward along the radial direction of the bottle mouth 101, and a total length of one of the anti-dismantle components a09 and a corresponding anti-dismantle part a01 along the radial direction of the bottle mouth 101 is greater than a distance between the inner wall of the outer thread sleeve b09 and the outer wall of the bottle body 100 around the bottle mouth 101. When the bottle plug 109 is inserted into the bottle mouth 101, the anti-dismantle components a09 are disposed under the anti-dismantle parts a01, the limit grooves b01 penetrate through the anti-dismantle parts a01 along the axial direction of the bottle mouth 101, and a width of each limit component c09 is adapted to a width of a corresponding limit groove b01. The radial direction of the bottle mouth 101 generally refers to the direction of the radius of the bottle mouth 101. By setting annular protrusions as the anti-dismantle components a09 and the anti-dismantle parts a01 and setting the limit grooves b01, an interference fit can be better achieved, thereby further enhancing the stability of the connection and the limit effect between the bottle plug 109 and the bottle mouth 101, while also facilitating the molding of the bottle body 100 and the bottle plug 109.

Referring to FIG. 1 to FIG. 9, in a specific embodiment, the bottle plug 109 includes a plug body 11 which is tube-shaped, the plug body 11 is in sealed fit with an inner wall of the bottle body 100 around the bottle mouth 101, the connecting component 113 is a connecting tube inserted into and connected to the plug body 11, the connecting component 113 defines an installation through hole 1131, the input end of the connecting component 113 is configured to connect to the external air supply device in a plug-in manner, an air input end 119 of the atomizing core 104 is installed on an output end of the installation through hole 1131 in a plug-in manner, and the spray outlet channel 112 is defined between an inner wall of the plug body 11 and an outer wall of the connecting component 113. The above settings make the bottle plug 109 and the connecting component 113 compact and conveniently connected, facilitating installation and maintenance.

In a specific embodiment, the connecting component 113 is connected to the plug body 11 through multiple connecting ribs 113a, the liquid outlet hole 105 of the liquid conveying channel 107 and the air outlet hole 106 of the air conveying channel 108 are disposed under the multiple connecting ribs 113a, the spray outlet channel 112 passes through the multiple connecting ribs 113a, coaxially surrounds the connecting component 113, and connects an inside of the bottle body 100 to an outside of the bottle body 100, the air outlet hole 106 and the liquid outlet hole 105 are spaced apart by a preset distance, axes of the air outlet hole 106 and the liquid outlet hole 105 intersect, and the air outlet hole 106 is configured to blow the air flow from top to bottom to an outer end surface of the liquid outlet hole 105. By setting the multiple connecting ribs 113a, the structural strength of the bottle plug 109 is enhanced, and the path of the spray outlet channel 112 is optimized, thereby improving the atomization effect. On the other hand, the spray outlet channel 112, the connecting component 113, the connecting ribs 113a, and the main body of the plug body 11 are more easily formed as an integrated unit.

Referring to FIG. 1 to FIG. 6, in a specific embodiment, the atomizing core 104 is provided with a tube inserting sleeve 115, the another end of the liquid sucking tube 116 is inserted into the tube inserting sleeve 115 and connected to the liquid conveying channel 107, and the end of the liquid sucking tube 116 is connected to a fragrance filter 117 in a plug-in manner. The fragrance filter 117 is connected to the end of the liquid sucking tube 116 and configured to filter impurities from the liquid fragrance. By setting the tube inserting sleeve 115 and the fragrance filter 117, the efficiency of liquid fragrance delivery is improved and the service life of the atomizing core 104 is extended, while also facilitating the installation of the fragrance filter 117.

Referring to FIG. 1 to FIG. 9, the disclosure further provides a fragrance diffuser. The fragrance diffuser includes an atomizing head 200 and the liquid storage bottle. The atomizing head 200 is provided with an air output end 209, the air output end 209 is detachably connected to the connecting component 113, the atomizing head 200 is provided with a connecting head 206, the connecting head 206 is configured to connect to the external air supply device, and the connecting head 206 is connected to the air conveying channel 108 through the air output end 209 and the connecting component 113. The atomizing head 200 of the fragrance diffuser is provided with the air output end 209 and the connecting head 206, the air output end 209 is detachably connected to the connecting component 113, and the connecting head 206 is configured to connect to the external air supply device, thereby achieving convenient connection between the fragrance diffuser and the liquid storage bottle.

Referring to FIG. 1 to FIG. 9, in a specific embodiment, the atomizing head 200 includes a shell 201, and the shell 201 defines a spray outlet chamber 203 and a spray outlet nozzle 202. The spray outlet nozzle 202 is connected to the spray outlet chamber 203. An inside wall of the shell 201 is provided with connecting threads 204, and an outer wall of the bottle body 100 around the bottle mouth 101 is provided with outer threads 1011 or an outer wall of the bottle plug 109 is provided with outer threads b091 to match with the connecting threads 204. The connecting component 113 is the installation through hole 1131 defined on the bottle plug 109, and the installation through hole 1131 is coaxial with the outer threads 1011/b091. The air output end 209 is tube-shaped and coaxial with the connecting threads 204. When the connecting threads 204 match with the outer threads 1011/b091, an end of the air output end 209 is inserted into the installation through hole 1131. The input end of the connecting component 113 is configured to connect to the external air supply device in a plug-in manner, and an air input end 119 of the atomizing core 104 is tube-shaped and installed on an output end of the installation through hole 1131 in a plug-in manner. A plug-in connection within the installation through-hole can be used and thereby facilitates the installation of an air supply system and reduces the number of installation steps. Through the design of the shell 201 and the connecting threads 204, a stable connection between the atomizing head 200 and the liquid storage bottle is achieved.

Referring to FIG. 1 to FIG. 6, in a specific embodiment, the liquid outlet hole 105 of the liquid conveying channel 107 and the air outlet hole 106 of the air conveying channel 108 are disposed in an inside of the bottle body 100, and the spray outlet channel 112 is connected to the inside of the bottle body 100 and the spray outlet chamber 203 individually. The atomized fragrance is output to the spray outlet chamber 203 and further processed by the atomizing head 200, enhancing the atomization effect and ensuring that the atomized fragrance can be smoothly output.

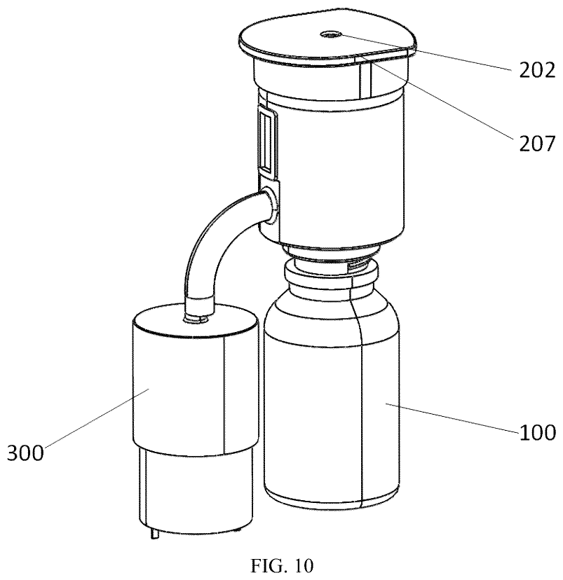

Referring to FIG. 1 to FIG. 6 and FIG. 10, in a specific embodiment, the connecting head 206 is elastic, an outer wall of the connecting head 206 protrudes outward relative to the shell 201, the fragrance diffuser further includes an air pump 300 as the external air supply device, the air pump 300 is configured to provide the air flow to the atomizing core 104 through the connecting head 206, the air conveying channel 108 penetrates through the air output end 209, an air inlet hole 1081 of the air conveying channel 108 penetrates through the shell 201, and the connecting head 206 is detachably installed on the air inlet hole 1081 in a plug-in manner to form sealed fit. Since the connecting head 206 is elastic, the connecting head 206 abuts against an output end of the air pump 300, allowing for a more flexible and convenient installation.

Referring to FIG. 1 to FIG. 6, in a specific embodiment, the atomizing head 200 further includes an atomizing filter 205, the atomizing filter 205 is installed inside the spray outlet chamber 203, an input end of the atomizing filter 205 is connected to the spray outlet chamber 203, a top of the shell 201 is threadedly connected to a cover 207, the cover 207 is configured to seal an opening 2011 of the shell 201, and the spray outlet nozzle 202 is defined on the cover 207 and connected to an output end of the atomizing filter 205. By setting the atomizing filter 205, the atomized fragrance is further purified, and the output quality of the fragrance diffuser is thereby enhanced. Meanwhile, the design of the cover 207 and the opening 2011 of the shell 201 facilitates the installation of the atomizing filter 205.

Referring to FIG. 1 to FIG. 6, in a specific embodiment, a top of the bottle plug 109 is in a sealing connection with the shell 201 through a sealing pad 208. The sealing pad 208 enhances the seal between the bottle plug 109 and the shell 201, preventing leakage of the liquid fragrance and the atomized fragrance.

In the disclosure, unless otherwise specified and limited, a first feature is “on” or “under” a second feature, it may be a direct contact between the first and second features, or an indirect contact between the first and second features through an intermediate medium. Moreover, the first feature being “on” or “above” the second feature may indicate that the first feature is directly above or diagonally above the second feature, or simply that the first feature is horizontally higher than the second feature. The first feature being “under” or “below” the second feature may indicate that first feature is located directly or diagonally below the second feature, or simply indicate that the first feature has a lower horizontal height than the second feature.

In the description of this specification, the reference terms such as “illustrated embodiments”, “another embodiment”, “other embodiments”, or “specific examples” means that the specific features, structures, materials, or characteristics described in conjunction with the embodiments or examples are included in at least one embodiment or example of the disclosure. In the specification, the schematic expressions of the above terms do not necessarily refer to the same embodiments or examples. Moreover, the specific features, structures, materials, or characteristics described can be combined in any one or more embodiments or examples in an appropriate manner. In addition, those skilled in the art can combine the different embodiments or examples described in this specification, as well as the features of different embodiments or examples, without conflicting with each other.

Although the embodiments of the disclosure are shown and described above, it can be understood that the above embodiments are exemplary and should not be construed as limiting the disclosure. Those skilled in the art may make changes, amendments, substitutions, and variations to the above embodiments within the scope of the disclosure.

What is claimed is:

1. A liquid storage bottle, comprising:
a bottle body (100), defining a bottle mouth (101) and a liquid storage chamber (102), wherein the bottle mouth (101) is connected to the liquid storage chamber (102), and the liquid storage chamber (102) is configured to store liquid fragrance; and
a bottle plug (109), inserted into the bottle mouth (101) to form sealed fit therewith;
wherein the bottle plug (109) is provided with an atomizing core (104), a liquid sucking tube (116) and a connecting component (113); the atomizing core (104) defines a liquid conveying channel (107) and an air conveying channel (108), and a liquid outlet hole (105) of the liquid conveying channel (107) and an air outlet hole (106) of the air conveying channel (108) are defined above a liquid surface; an input end of the connecting component (113) is configured to be connected to an external air supply device, an output end of the connecting component (113) is connected to the air conveying channel (108), and the connecting component (113) is configured to provide an air flow to the atomizing core (104); an end of the liquid sucking tube (116) is inserted into the liquid storage chamber (102), another end of the liquid sucking tube (116) is connected to the liquid conveying channel (107), and the liquid sucking tube (116) is configured to convey the liquid fragrance in the liquid storage chamber (102) to the atomizing core (104); the atomizing core (104) is configured to mix and atomize the air flow provided by the connecting component (113) and the liquid fragrance provided by the liquid sucking tube (116); and the bottle plug (109) defines a spray outlet channel (112).

2. The liquid storage bottle as claimed in claim 1, wherein the bottle plug (109) is detachably inserted into the bottle mouth (101) to form sealed fit therewith; or
the bottle plug (109) is provided with anti-dismantle components (a09), the bottle body (100) is provided with anti-dismantle parts (a01) around the bottle mouth (101), and when the bottle plug (109) is inserted into the bottle mouth (101), the anti-dismantle components (a09) are engaged with the anti-dismantle parts (a01).

3. The liquid storage bottle as claimed in claim 2, wherein the bottle plug (109) comprises an upper plug body (110) and a lower plug body (111), the lower plug body (111) is inserted into the bottle mouth (101) and in a sealing connection with the bottle mouth (101) through a sealing ring (114), an outer wall of the bottle body (100) around the bottle mouth (101) is provided with outer threads (1011), and the bottle body (100) is fixed with an atomizing head (200) at the bottle mouth (101) through the outer threads (1011); or
the bottle plug (109) comprises an inner plug body (d09) and an outer thread sleeve (b09), the outer thread sleeve (b09) is sleeved on the outer wall of the bottle body (100) around the bottle mouth (101), an outside of the outer thread sleeve (b09) is provided with outer threads (b091), the outer thread sleeve (b09) is fixed with the atomizing head (200) through the outer threads (b091), the inner plug body (d09) is inserted into the bottle mouth (101), and upper ends of the inner plug body (d09) and the outer thread sleeve (b09) are connected to each other and are in a sealing connection with a top of the bottle body (100) around the bottle mouth (101) through the sealing ring (114).

4. The liquid storage bottle as claimed in claim 1, wherein the bottle plug (109) comprises an inner plug body (d09) and an outer thread sleeve (b09), the outer thread sleeve (b09) is sleeved on an outer wall of the bottle body (100) around the bottle mouth (101), an inner wall of the outer thread sleeve (b09) is provided with anti-dismantle components (a09), the outer wall of the bottle body (100) around the bottle mouth (101) is provided with an anti-dismantle part (a01), the anti-dismantle components (a09) are disposed corresponding to the anti-dismantle parts (a01), and the outer thread sleeve (b09) is fixed on the bottle body (100) around the bottle mouth (101) through an interference fit between the anti-dismantle components (a09) and the anti-dismantle parts (a01).

5. The liquid storage bottle as claimed in claim 4, wherein the inner wall of the outer thread sleeve (b09) is provided with limit components (c09), the outer wall of the bottle body (100) around the bottle mouth (101) defines limit grooves (b01), the limit components (c09) and the limit grooves (b01) extend along an axial direction of the bottle mouth (101), when the bottle plug (109) is inserted into the bottle mouth (101), the limit components (c09) are inserted into the limit grooves (b01) respectively along the axial direction of the bottle mouth (101), and the limit components (c09) and the limit grooves (b01) are configured to limit rotating of the bottle plug (109) relative to the bottle mouth (101).

6. The liquid storage bottle as claimed in claim 5, wherein the anti-dismantle components (a09) and the anti-dismantle parts (a01) are protrusions disposed annularly, the anti-dismantle components (a09) extend inward along a radial direction of the bottle mouth (101), the anti-dismantle parts (a01) extend outward along the radial direction of the bottle mouth (101), a total length of one of the anti-dismantle components (a09) and a corresponding one of the anti-dismantle parts (a01) along the radial direction of the bottle mouth (101) is greater than a distance between the inner wall of the outer thread sleeve (b09) and the outer wall of the bottle body (100) around the bottle mouth (101), when the bottle plug (109) is inserted into the bottle mouth (101), the anti-dismantle components (a09) are disposed under the anti-dismantle parts (a01), the limit grooves (b01) penetrate through the anti-dismantle parts (a01) along the axial direction of the bottle mouth (101), and a width of each of the limit components (c09) is adapted to a width of a corresponding one of the limit grooves (b01).

7. The liquid storage bottle as claimed in claim 1, wherein the bottle plug (109) comprises a plug body (11) which is tube-shaped, the plug body (11) is in sealed fit with an inner wall of the bottle body (100) around the bottle mouth (101), the connecting component (113) is a connecting tube inserted into and connected to the plug body (11), the connecting component (113) defines an installation through hole (1131), the input end of the connecting component (113) is configured to connect to the external air supply device in a plug-in manner, an air input end (119) of the atomizing core (104) is installed on an output end of the installation through hole (1131) in a plug-in manner, and the spray outlet channel (112) is defined between an inner wall of the plug body (11) and an outer wall of the connecting component (113).

8. The liquid storage bottle as claimed in claim 7, wherein the connecting component (113) is connected to the plug body (11) through a plurality of connecting ribs (113a), and the liquid outlet hole (105) of the liquid conveying channel (107) and the air outlet hole (106) of the air conveying channel (108) are disposed under the plurality of connecting ribs (113a); the spray outlet channel (112) passes through the plurality of connecting ribs (113a), coaxially surrounds the connecting component (113), and connects an inside of the bottle body (100) to an outside of the bottle body (100); and the air outlet hole (106) and the liquid outlet hole (105) are spaced apart by a preset distance, axes of the air outlet hole (106) and the liquid outlet hole (105) intersect, and the air outlet hole (106) is configured to blow the air flow from top to bottom to an outer end surface of the liquid outlet hole (105).

9. The liquid storage bottle as claimed in claim 1, wherein the atomizing core (104) is provided with a tube inserting sleeve (115), the another end of the liquid sucking tube (116) is inserted into the tube inserting sleeve (115) and connected to the liquid conveying channel (107), and the end of the liquid sucking tube (116) is connected to a fragrance filter (117) in a plug-in manner.

10. A fragrance diffuser, comprising: an atomizing head (200), and the liquid storage bottle as claimed in claim 1;
wherein the atomizing head (200) is provided with an air output end (209), the air output end (209) is detachably connected to the connecting component (113), the atomizing head (200) is provided with a connecting head (206), the connecting head (206) is configured to connect to the external air supply device, and the connecting head (206) is connected to the air conveying channel (108) through the air output end (209) and the connecting component (113).

11. The fragrance diffuser as claimed in claim 10, wherein the atomizing head (200) comprises a shell (201), the shell (201) defines a spray outlet chamber (203) and a spray outlet nozzle (202), the spray outlet nozzle (202) is connected to the spray outlet chamber (203), an inside wall of the shell (201) is provided with connecting threads (204), an outer wall of the bottle body (100) around the bottle mouth (101) is provided with outer threads (1011) or an outer wall of the bottle plug (109) is provided with outer threads (b091) to match with the connecting threads (204), the connecting component (113) is an installation through hole (1131) defined on the bottle plug (109), the installation through hole (1131) is coaxial with the outer threads (1011/b091), the air output end (209) is tube-shaped and coaxial with the connecting threads (204), when the connecting threads (204) match with the outer threads (1011/b091), an end of the air output end (209) is inserted into the installation through hole (1131), the input end of the connecting component (113) is configured to connect to the external air supply device in a plug-in manner, and an air input end (119) of the atomizing core (104) is tube-shaped and installed on an output end of the installation through hole (1131) in a plug-in manner.

12. The fragrance diffuser as claimed in claim 11, wherein the liquid outlet hole (105) of the liquid conveying channel (107) and the air outlet hole (106) of the air conveying channel (108) are disposed in an inside of the bottle body (100), and the spray outlet channel (112) is connected to the inside of the bottle body (100) and the spray outlet chamber (203) individually.

13. The fragrance diffuser as claimed in claim 10, wherein the connecting head (206) is elastic, an outer wall of the connecting head (206) protrudes outward relative to the shell (201), the fragrance diffuser further comprises an air pump (300) as the external air supply device, the connecting head (206) abuts against an output end of the air pump (300), the air pump (300) is configured to provide the air flow to the atomizing core (104) through the connecting head (206), the air conveying channel (108) penetrates through the air output end (209), an air inlet hole (1081) of the air conveying channel (108) penetrates through the shell (201), and the connecting head (206) is detachably installed on the air inlet hole (1081) in a plug-in manner to form sealed fit.

14. The fragrance diffuser as claimed in claim 10, wherein the atomizing head (200) further comprises an atomizing filter (205), the atomizing filter (205) is installed inside the spray outlet chamber (203), an input end of the atomizing filter (205) is connected to the spray outlet chamber (203), a top of the shell (201) is threadedly connected to a cover (207), the cover (207) is configured to seal an opening (2011) of the shell (201), and the spray outlet nozzle (202) is defined on the cover (207) and connected to an output end of the atomizing filter (205).

15. The fragrance diffuser as claimed in claim 10, wherein a top of the bottle plug (109) is in a sealing connection with the shell (201) through a sealing pad (208).
